(19) United States
(12) Patent Application Publication
(10) Pub. No.: US 2006/0247653 A1
(43) Pub. Date: Nov. 2, 2006

(54) Sleeve, drill and puncture instrument

(75) Inventors: Dan Akerfeldt, Uppsala (SE); Eva Nicklasson, Uppsala (SE)

Correspondence Address: FOLEY AND LARDNER LLP;SUITE 500, 3000 K STREET NW, WASHINGTON, DC, 20007, US

(73) Assignee: RADI MEDICAL SYSTEMS AB

(21) Appl. No.: 11/412,110

(22) Filed: Apr. 27, 2006

Related U.S. Application Data
(60) Provisional application No. 60/675,468, filed on Apr. 28, 2005.

(51) Int. Cl. A61B 17/60

(52) U.S. Cl. 606/96

(57) ABSTRACT

A surgical puncture instrument (30) includes a drill (31) and a cutter sleeve (36). The sleeve is shaped to continue the shape of the drill so that the sleeve participates in the drilling operation. This permits the sleeve to enter hard tissue and allows the sleeve to be used for access to inner tissue to, for example, obtain a biopsy with a biopsy needle (13).

Priority is hereby claimed to U.S. Provisional Application 60/675,468, filed Apr. 28, 2005, whose entire contents are incorporated herein by reference.

### FIELD OF THE INVENTION

The present invention relates to a device for the sampling of tissue from a human or animal being, and in particular to a puncture instrument comprising a needle and a sleeve, which together work as a drill.

### BACKGROUND OF THE INVENTION

One way to gain access to a lesion through a cortical bone is to introduce a conventional spiral drill equipped with a cannula, drill through the cortical bone, and then remove the drill. The cannula remains in place in the soft tissue, but cannot be inserted through the drilled hole in the cortical bone, because the outer diameter of the cannula is larger than the diameter of the drilled hole. A disadvantage is that the cannula easily can move out of position, making it difficult to relocate the drilled hole in order to, for example, introduce a biopsy needle.

The above problem is addressed in the U.S. Pat. No. 5,810,826, which is assigned to the present assignee and which discloses an instrument for puncturing a cortical bone. The entire contents of this patent are incorporated herein by reference, for details of this technology and associated medical procedures. The instrument comprises a cannula, the distal end of which is externally tapered and whose proximal end is provided with a handle. The instrument comprises further a needle or stylet, which is axially movable inside the cannula. The needle has a drill tip whose drill point has radial displacement in relation to the axial centre of the needle. The effect is that with this instrument a hole can be drilled which has a diameter that is larger than the diameter of the cannula. With such a large diameter hole, the cannula along with the drill can easily enter the drilled hole.

Although the technology described in the above patent represents a progress in comparison with the traditional procedure, it is still accompanied by some disadvantages. A drill or needle having an eccentric drill tip is, for example, more expensive to manufacture than a conventional drill with a concentric drill tip. From more technical aspects, a drill hole having a diameter corresponding to the eccentricity of the drill tip, i.e. being larger than the diameter of the drill itself, is not produced at the exit portion of the drill hole, i.e. at the point where the drill tip penetrates through e.g. a cortical bone and enters the marrow present therein. Further, for sharp needles or drills having an acute point angle, the eccentricity of the drill or needle does not work in an optimal way; and for certain punctures which require needles with a small point angle, e.g., when drilling a hole at an angle that is small (much less than 90°) relative to the surface of the tissue (usually bone) of interest, an eccentric drill is therefore not very suitable. It can also be appreciated that there always is a potential risk that the cannula cannot be advanced into a drill hole created in hard tissue such as the cortical bone, because remainders such as drillings from the drilling procedure are wedged between the inner surface of the drill hole and the mantle of the cannula, thereby making it impossible to advance the cannula all the way to the end of the drill hole.

### SUMMARY OF THE INVENTION

A general object of the present invention is to provide an improved design for a medical puncture instrument adapted for the puncturing of hard tissue and comprising a needle and a sleeve, which puncture instrument ensures proper positioning of the sleeve within a puncture created by the puncture instrument.

According to the present invention, a medical puncture instrument comprises a sleeve and a needle, which is axially movable inside the sleeve. The proximal end of the needle is provided with a handle, while the distal end of the needle is formed as one or several drill cutters. The distal end of the needle will thereby act as a drill tip, and has a point angle. The outer diameter of the needle is essentially equal to the inner diameter of the sleeve. The proximal end of the sleeve is provided with a head, against which the needle handle stops when the needle is inserted into the sleeve. The distal end of the sleeve is tapered, with essentially the same angle as the point angle of the needle tip. The length of the sleeve is adapted to the length of the needle in such a way that when the sleeve is positioned over the needle, the tapered distal end of the sleeve coincides with the proximal end of the cutters. Further, the distal portion of the needle is provided with one or several grooves or recesses, which can be slightly helical, as is normal for a drill. The distal portion of the sleeve is provided with cuts whose shape coincides with the shape of the grooves or recesses in the needle. The cuts in the sleeve can thereby be regarded as a continuation of the grooves in the needle. From the above, it should now be clear that the needle and the sleeve together co-operate to act as a puncture instrument in the form of a drill, i.e. the sleeve participates in the drilling operation.

When a drilling operation is completed, typically when the distal end of the needle has penetrated through the cortical bone, the needle is withdrawn, while the sleeve is left in the drill hole. A sampling device, such as a biopsy needle, can then be advanced through the sleeve to take a sample of the tissue, e.g. marrow, located within the hard tissue, e.g. the cortical bone. Since the sleeve constitutes a part of the drill, no advancement of the sleeve is necessary, and access to the tissue of interest is thereby guaranteed.

### BRIEF DESCRIPTION OF THE DRAWINGS

FIGS. 1a and 1b show the distal ends of a needle and a sleeve, respectively, which together form a puncture instrument according to one embodiment of the present invention.

FIGS. 2a, 2b and 2c illustrate the basic steps of a puncture operation in which the puncture instrument according to one embodiment of the present invention may be used.

FIGS. 3a-e are different views of an embodiment of a puncture instrument according to the present invention.

### DETAILED DESCRIPTION OF PREFERRED EMBODIMENTS

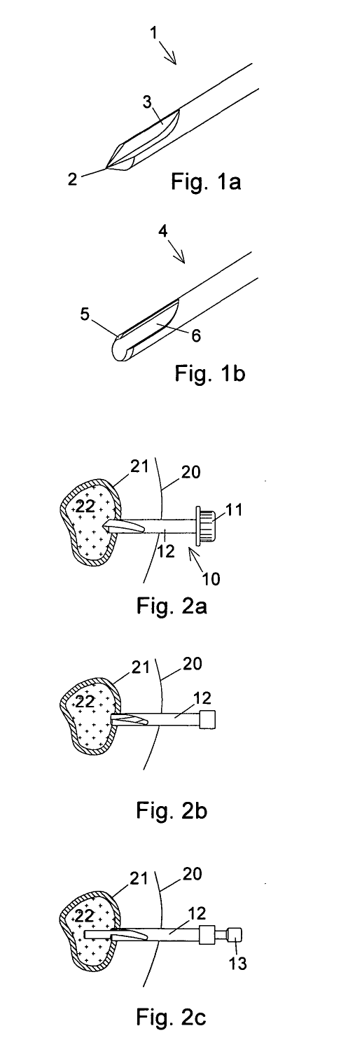

The basic elements of a puncture instrument according to the present inventions are illustrated in FIGS. 1a and 1b, wherein FIG. 1a shows a distal portion of a needle (or drill) 1, which is provided with a drill tip 2 and a groove 3 (sometimes called a flute), and wherein FIG. 1b shows a distal portion of a sleeve 4, which is provided with a chamfered distal end 5 and a cut (or opening or recess) 6. The inner diameter of the sleeve 4 is closely adapted to the outer diameter of the needle 1. Basically, the needle 1 can be regarded as a conventional drill 1, whereas it is the special features of the sleeve 4—when co-operating with the needle 1—that accomplish a puncture instrument according to this embodiment of the invention. The drill tip 2 of the needle 1 has an angle, which preferably is the same, or almost the same, as the chamfering of the distal end 5 of the sleeve 4. Moreover, the dimensions of the recess 6 in the sleeve 4 are adapted to the dimensions of the groove 3 in the needle 1 such that the recess continues the groove of the drill. It should therefore be clear that when the needle 1 has been inserted into the sleeve 4, such that distal end 5 of the sleeve 4 coincides with the proximal end of the angled drill tip 2 of the needle 1, the sleeve 4 will effectively constitute the outer mantle of a composite drill, which constitutes the puncture instrument according to this embodiment of the present invention.

FIGS. 2a, 2b, and 2c illustrate schematically the advantage of providing a puncture instrument in the form of a drill whose outer mantle is arranged as a separate sleeve. More specifically, FIG. 2a shows a first step in a puncturing operation, wherein a puncture instrument 10, which comprises a needle (or drill) 11 and a sleeve 12, has penetrated through soft tissue 20 overlaying a hollow bone 21 such that the tip of the needle 11 has drilled through the bone 21 and entered into the marrow 22 located within the hollow bone 21. A second step of the puncturing operation is illustrated in FIG. 2b, where the needle 11 of the puncture instrument 10 has been retracted out of the sleeve 12, to thereby leave only the sleeve 12 in place. FIG. 2c illustrates how a biopsy needle 13 has been inserted into the sleeve 12, such that the distal end of the biopsy needle 13 has access to the marrow 22, wherefrom a sample now can be taken.

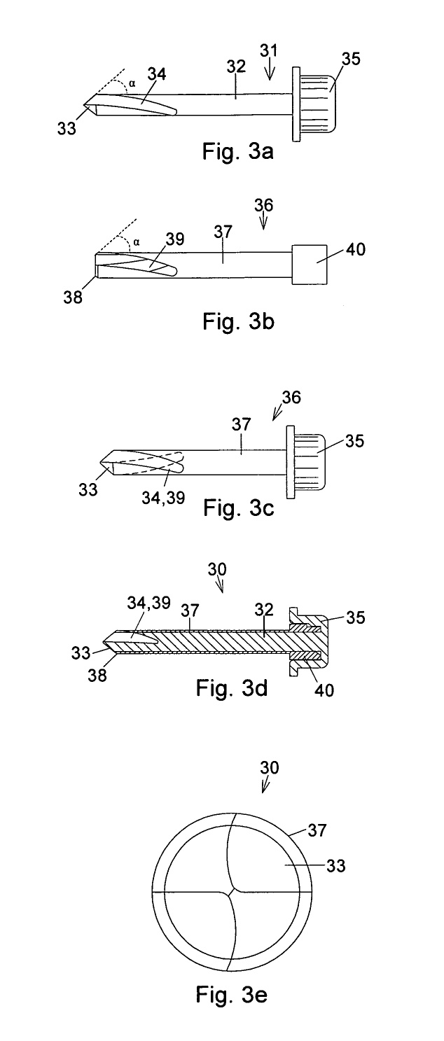

In FIGS. 3a-3e an embodiment of a puncture instrument 30 according to the present invention is illustrated. The puncture instrument 30 comprises a needle or drill 31, which is separately shown in FIG. 3a. The needle 31 has an elongated body 32, whose distal end is provided with a drill tip 33. The distal portion of the needle 31 has been provided with two grooves (or flutes) 34, of which only one is visible in FIG. 3a, which extend from the drill tip 33 a distance proximally in the elongated body 32 of the needle 31. In this embodiment, the grooves 34 are slightly curved around the longitudinal axis of the needle 31, but similar grooves that extend in straight lines along a distal portion of a needle and other shapes suitable for drilling are also possible. The proximal end of the needle 31 is provided with a handle 35. When taken by itself, the needle 31 can be regarded as a standard drill; and the skilled person will realize that many modifications could be done regarding the shape of the drill and drill tip, including the different angles provided there. In FIG. 3a, one angle of the drill tip 33 has been marked with a. Also the shape and dimensions of the grooves could be varied, as is normal for this type of puncture drills. It may in particular be noted that only one drill cutter can be provided and consequently only one groove, but also more than two drill cutters and more than two grooves could be provided.

FIG. 3b shows a sleeve 36, which also constitutes part of the puncture instrument 30. The sleeve 36 has an elongated body 37, with chamfered distal end 38 and two recesses (or cuts or openings) 39, which extend from the chamfered distal end 38 a distance proximally in the elongated body 37 of the sleeve 36. In this particular embodiment, the recesses extend in a helical shape. The proximal end of the sleeve 36 is provided with an enlarged head 40. The inner diameter of the sleeve 36 is adapted to the outer diameter of the needle 31, such that the needle 31 can be inserted into the sleeve 36. The chamfering of the distal end 38 of the sleeve 36 is advantageously adapted to the angle a of the drill tip 33, as is indicated in FIG. 3a and FIG. 3b. The length of the sleeve 36 is adapted to the length of the needle 31 such that when the needle 31 is inserted into the sleeve 36, the chamfered distal end 38 of the sleeve 36 is approximately even with the proximal end of the drill tip 33, as is best seen in FIG. 3d. The sleeve 36 may contain one or more cutting edges that are extensions of cutting edge(s) of needle 31. Further, the interior of the handle 35 of the needle 31 and the exterior of a head 40 of the sleeve 36 are shaped to co-operate such that when the needle 31 is inserted into the sleeve 36, the needle 31 cannot rotate inside the sleeve 36, and neither can the sleeve 36 rotate around the needle 31. In practice, it is possible to provide the interior of the handle 35 and the exterior of the head 40 with any kind of keying that prevents rotational movement and/or longitudinal movement of the sleeve 36 relative to the needle 31 and/or correctly positions grooves 34 with respect to recesses 39. The needle 31 is releasable from the sleeve 36.

In FIGS. 3c, 3d, and 3e, the needle 31 and the sleeve 36 are shown in an assembled state, wherein these two members together form the puncture instrument 30. It should in particular be clear that the needle 31 and the sleeve 36 together form a drill 30, which during a drill operation works as a conventional drill, but whose outer mantle is made from the separate sleeve, which can be left in the puncture hole, to achieve the advantages discussed above. From FIG. 3e it can in particular be noted that the inner diameter of the sleeve 36 is adapted to the outer diameter of the needle 31.

As indicated above, a drill tip of a needle can be provided with any number of drill cutters and therefore any number of grooves could be provided in the distal portion of the needle; and a mating sleeve could therefore be provided with any number of corresponding recesses. It should, however, be noted that the more recesses, the less amount of material will remain at the distal portion of the sleeve, which thereby will be weaker. The number of drill cutters and therefore grooves and recesses should be adapted to the intended purpose of the corresponding puncture instrument. Also the wall thickness of the sleeve should be adapted to the puncture operation to be practiced.

General drill design, structure, and terminology is set forth in “Orthopaedic Bone Drills—Can They Be Improved?: Temperature Changes Near the Drilling Face” Colin Natali et al., The Journal of Bone & Joint Surgery, May 1996, pp. 357-362 and in Unit 38 of Machine Tool Technology, Victor E. Repp et al., Glencoe Publishing Co. (1984), ISBN 0-02-671570-8, whose entire contents are incorporated herein by reference.

Although the present invention has been described with reference to specific embodiments, also shown in the appended drawings, it will be apparent for those skilled in the art that many variations and modifications can be done within the scope of the invention as described in the specification and defined with reference to the claims below. It should in particular be noted that an ordinary drill could be used as a needle in the puncture instrument, i.e. in practice only a sleeve has to be adapted to an existing drill, to produce a puncture instrument according to the present invention. Some of the claims below are consequently directed to such a sleeve alone. Also, in the embodiments above, a flute of the overall puncture instrument (that is, a pathway for material being removed by the cutting edge(s)) is formed by a combination of an opening in the sleeve and the flute of the drill; however, the flute of the overall puncture instrument may be formed entirely in the sleeve such that no flute is formed in the drill.

What is claimed is:

1. A sleeve for use with a surgical drill, the sleeve comprising:
an elongated body configured to fit over a surgical drill; and
an opening, in the elongated body, shaped to continue a flute of the drill.

2. A sleeve as set forth in claim 1, further comprising:
a member shaped to mate with the drill to rotationally fix the sleeve with respect to the drill.

3. A sleeve as set forth in claim 1, further comprising:
a chamfered distal end chamfered to continue an angled drill tip of the drill.

4. A sleeve as set forth in claim 1, wherein the opening extends in a helical shape along the elongated body.

5. A sleeve as set forth in claim 1, further comprising:
a member shaped to mate with the drill to fix the sleeve to the drill in a direction parallel to a central axis of the elongated body.

6. A sleeve as set forth in claim 1, further comprising at least one cutting edge shaped as an extension of a cutting edge of the drill.

7. A sleeve as set forth in claim 2, wherein the sleeve and drill are fixed such that the drill is releasable from the sleeve.

8. A sleeve as set forth in claim 5, wherein the sleeve and drill are fixed such that the drill is releasable from the sleeve.

9. A puncture instrument comprising:
a drill; and
a sleeve as set forth in claim 1.

10. A puncture instrument comprising:
a drill; and
a sleeve as set forth in claim 2.

11. A puncture instrument as set forth in claim 9, wherein an outer diameter of the drill is substantially the same as an inner diameter of the sleeve.

12. A puncture instrument as set forth in claim 10, wherein an outer diameter of the drill is substantially the same as an inner diameter of the sleeve.

13. A surgical puncture instrument comprising:
a surgical needle having a drill tip at a distal end; and
a sleeve having an elongated body configured to fit over the surgical needle and a flute in the elongated body.
